(19) United States
(12) Patent Application Publication
(10) Pub. No.: US 2016/0310091 A1
(43) Pub. Date: Oct. 27, 2016

(54) MEDICAL DEVICE AND TREATMENT METHOD

(71) Applicant: TERUMO KABUSHIKI KAISHA, Tokyo (JP)

(72) Inventors: Katsuhiko Shimizu, Fujinomiya-city (JP); Keiko Yamashita, Atsugi-city (JP); Hiroki Hosono, Hadano-city (JP); Yasukazu Sakamoto, Hiratsuka-city (JP)

(21) Appl. No.: 15/134,216

(22) Filed: Apr. 20, 2016

(30) Foreign Application Priority Data
Apr. 22, 2015 (JP) 2015-087448

(51) Int. Cl. A61B 6/00

(52) U.S. Cl. CPC A61B 6/504; A61B 6/481

(57) ABSTRACT

A medical device and a treatment method capable of effectively suppressing an inflow of a contrast agent injected into blood vessels into kidney are provided. The medical device for suppressing an inflow of a contrast agent into kidney can include: an elongated inner tube, and a discharging portion configured to be capable of discharging a fluid in a proximal direction from both sides of a distal portion of the inner tube with an axial center being interposed therebetween, wherein the discharging portion includes a plurality of discharging holes arranged in one direction.

### CROSS REFERENCE TO RELATED APPLICATIONS

This application claims priority to Japanese Pat. App. No. 2015-087448, filed on Apr. 22, 2015, and entitled “MEDICAL DEVICE AND TREATMENT METHOD.”

### TECHNICAL FIELD

The present invention generally relates to a medical device and a treatment method for suppressing an inflow of a contrast agent injected into blood vessels into kidney.

### BACKGROUND

Percutaneous coronary intervention (PCI) developed for restoring a blood flow may be performed as a method of treatment of ischemic heart diseases such as angina pectoris and myocardial infarction. The PCI includes inserting a balloon into a stenosed site or an occluded site in coronary artery and dilating the balloon to forcedly widen the stenosed site or the occluded site. A contrast agent is administered to coronary artery, and then the PCI is performed while monitoring the stenosed site or the occluded site under radiography.

For patients having a renal dysfunction, the PCI may cause contrast-induced nephropathy due to side effects of the contrast agent, and in some cases, dialysis treatment may be required. The cause is not clearly defined, but one of conceivable causes is vascular factors such as renal ischemia caused by decreases of renal perfusion and glomerular filtration rate as a result of the contrast agent flowing into kidney and triggering renovascular contraction. In addition, it is also known that the contrast agent is directly cytotoxic for renal tubular cells.

Therefore, various methods for suppressing an inflow of a contrast agent into kidney are proposed. For example, U.S. Patent Application Publication No. 2014/0025037 describes a method of suppressing an inflow of a contrast agent into kidney by providing perfusion to left and right renal arteries after administration of the contrast agent.

The contrast agent is also used in intervention of artery of lower limb and Transcatheter Aortic Valve Implantation (TAVI), and similar toxic potential for kidney has become a problem.

### SUMMARY

A device disclosed in U.S. Patent Application Publication No. 2014/0025037provides perfusion into renal artery, but is not configured to prevent the inflow of the contrast agent into renal artery. Therefore, the contrast agent potentially flows into the renal artery together with the perfusion.

In order to solve the above-described problem, it is an object of the embodiments described herein to provide a medical device and a treatment method configured to effectively suppress an inflow of a contrast agent injected into blood vessels into kidney.

A medical device configured to achieve the aforesaid object can be a medical device for suppressing an inflow of the contrast agent into kidney, including: an elongated shaft portion; a discharging portion configured to be capable of discharging a fluid in a direction orthogonal to an axial center or in a proximal direction from both sides of a distal portion of the shaft portion with the axial center being interposed between the both sides of the distal portion, wherein the discharging portion includes a plurality of discharging holes arranged in one direction or discharging holes elongated in one direction in a slit shape.

With the medical device configured as described above discharges a fluid from the plurality of discharging holes arranged in one direction or the discharging holes elongated in one direction in a slit shape, the discharged fluid forms a film shape. With the discharge of the film-shaped fluid in the direction orthogonal to the axial center or in the proximal direction from both sides of the shaft portion with the axial center interposed therebetween, the fluid can be flowed so as to block off both entry portions of the two renal arteries from descending aorta into which the shaft portion is to be inserted while diluting the contrast agent with the fluid. Therefore, the inflow of the contrast agent into the renal arteries are effectively reduced. With the inflow of the contrast agent into the renal arteries reduced from occurring, an effect of the contrast agent on kidney is reduced.

With the shaft portion including an expandable portion configured to be capable of expanding radially outward, a position of the shaft portion can be fixed by bringing the expandable portion into contact with an intravascular wall surface, so that the inflow of the contrast agent into the renal arteries is reduced further effectively with a stabilized fluid discharging direction.

With the discharging portion being disposed at the expandable portion, the fluid can be discharged so as to flow from a position close to the blood vessel wall along a blood vessel wall. Therefore, the inflow of the contrast agent into the renal arteries is reduced further effectively by the film-shaped fluid.

With the medical device further including a detecting portion configured to detect the contrast agent, the fluid can be discharged at the same timing as the detection of the contrast agent. Therefore, the inflow of the contrast agent into the renal arteries is reduced further effectively.

With the shaft portion including an X-ray imaging marker at the distal portion thereof, the shaft portion can be positioned at an adequate position under radiography, and discharge of the fluid to a desirable position is enabled, so that the inflow of the contrast agent into the renal arteries is reduced further effectively.

A treatment method for achieving the aforesaid object is a treatment method for suppressing an inflow of a contrast agent into kidney including (i) an inserting step for inserting a medical device having a discharging portion configured to be capable of discharging a fluid at a distal portion of an elongated shaft portion into descending aorta; (ii) a providing step for providing the discharging portion on an upstream of an entry portion of renal artery; and (iii) a discharging step for discharging the fluid from the discharging portion in a film shape so as to cover the entry portion upon arriving of the contrast agent flowing from the upstream at the entry portion of the renal artery. According to the aforesaid treatment method, since the fluid is discharged in a film shape so as to cover the entry portion of the renal artery, the inflow of the contrast agent into renal artery is effectively reduced while diluting the contrast agent with the fluid. With the inflow of the contrast agent into the renal arteries reduced from occurring, an effect of the contrast agent on kidney is reduced.

With the discharging step including discharging the fluid from a discharging portion in a direction orthogonal to a axial center or in a proximal direction from both sides of a distal portion of the shaft portion with the axial center being interposed between the both sides of the distal portion, the film-shaped flow may be generated so as to block off both entry portions of the two renal arteries branched from the descending aorta. Therefore, the inflow of the contrast agent into the renal arteries is reduced.

With the providing step including fixing the position of the shaft portion by expanding an expandable portion configured to be capable of expanding radially outward in both directions from a distal portion of the shaft portion and bringing the expandable portion into contact with the descending aorta, the fluid discharging direction is stabilized by fixing the position of the shaft portion. Therefore, the inflow of the contrast agent into the renal arteries is reduced further effectively.

With the providing step including discharging the fluid from the discharging portion provided at the expandable portion, the fluid can be discharged so as to flow from the position close to the blood vessel wall along the blood vessel wall. Therefore, the film-shaped fluid suppresses the inflow of the contrast agent into the renal arteries further effectively.

With the discharging step including discharging the fluid from the discharging portion when the contrast agent is detected by a detecting portion configured to be capable of detecting the contrast agent and provided on the medical device, the fluid can be discharged at the same timing as the detection of the contrast agent. Therefore, the inflow of the contrast agent into the renal arteries is reduced further effectively.

With the providing step including providing the discharging portion while confirming a position of an X-ray imaging marker provided at the distal portion of the medical device under radiography, the discharging portion X can be disposed at an adequate portion and thus the fluid can be discharged to the desirable position, so that the inflow of the contrast agent into the renal arteries is reduced further effectively.

### BRIEF DESCRIPTION OF DRAWINGS

FIG. 1 is a plan view illustrating an embodiment of a medical device.

FIG. 2 is a vertical cross-sectional view of the medical device illustrating a state in which an expandable portion is expanded.

FIG. 3 is a vertical cross-sectional view of the medical device illustrating a state in which the expandable portion is contracted.

FIG. 4 is a cross-sectional view illustrating a state in which the medical device is inserted into descending aorta.

FIG. 5 is a cross-sectional view illustrating a state in which physiological salt solution is discharged from the medical device.

FIG. 6 is a flowchart illustrating a flow of control performed by a control unit.

FIG. 7 is a plan view illustrating an embodiment of a medical device.

FIG. 8 is a plan view illustrating a distal portion of the medical device.

FIG. 9 is a cross-sectional view illustrating the distal portion of the medical device.

FIG. 10 is a cross-sectional view illustrating a state in which a physiological salt solution is discharged from the medical device.

FIG. 11 is a schematic drawing illustrating the state in which the physiological salt solution is discharged from the medical device.

FIG. 12 is a schematic drawing illustrating a state in which a physiological salt solution is discharged from the medical device toward renal arteries.

FIG. 13 is a plan view illustrating an embodiment of a modification of the medical device.

FIG. 14 is a plan view illustrating an embodiment of another modification of the medical device.

### DETAILED DESCRIPTION

Embodiments of the invention will be described with reference to the drawings below. Note that dimensional ratios of the drawings may be exaggerated and thus may be different from actual ratios for the sake of convenience of description. In this specification, a side of the device which is inserted into blood vessels is referred to as “distal side” and a hand side to be operated is referred to as “proximal side”.

A medical device 10 can be a device configured to suppress an inflow of a contrast agent injected into blood vessels into renal arteries from descending aorta in a high-concentration state.

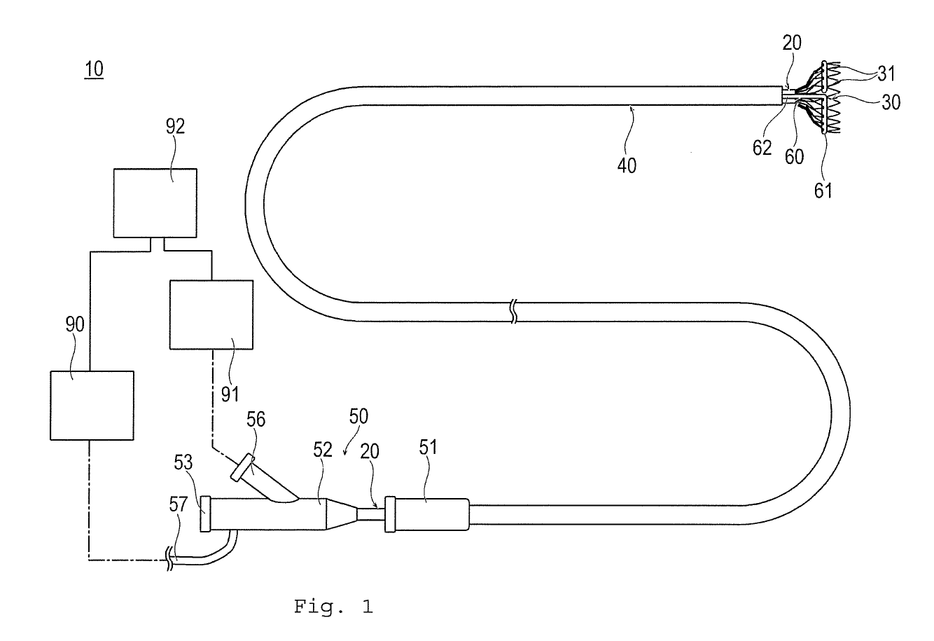

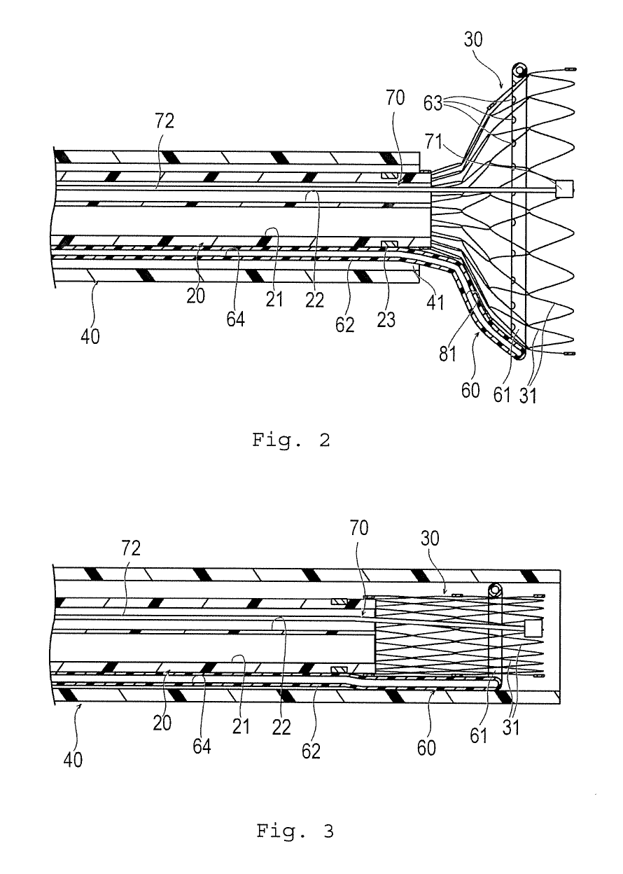

The medical device 10 can include: an inner tube 20 (shaft portion); an outer tube 40 configured to allow storage of the inner tube 20 in an interior thereof, an expandable portion 30 configured to be expandable and contractable at a distal portion of the inner tube 20; a discharging portion 60 configured to discharge a fluid; a detecting portion 70 configured to detect a contrast agent; and an operating unit 50 for operating the expandable portion 30, which may be as illustrated in FIGS. 1 and 2.

The inner tube 20 is an elongated tubular member and includes an insertion lumen 21 formed to allow insertion of a guide wire W in the interior thereof, and a wiring lumen 22 in which the detecting portion 70 is disposed. The insertion lumen 21 and the wiring lumen 22 open at a distal end portion of the inner tube 20. An X-ray imaging marker 23 having an X-ray contrasting property is disposed at the distal portion of the inner tube 20. The expandable portion 30 is interlocked with an outer peripheral surface of the distal portion of the inner tube 20. A proximal portion of the inner tube 20 is interlocked with a second operating unit 52 which constitutes part of the operating unit 50.

The outer tube 40 is an elongated tubular member configured to allow storage of the inner tube 20 therein, and to be movable in an axial direction relatively with respect to the inner tube 20. The outer tube 40 opens at an outer tube opening 41 formed on a distal side, and a proximal portion of the outer tube 40 is interlocked with a first operating unit 51 which constitutes part of the operating unit 50.

The expandable portion 30 is interlocked with the outer peripheral surface of the distal portion of the inner tube 20, and includes wire members 31 formed of elastically deformable wires and forming a net shape so as to have a plurality of void portions. The expandable portion 30 is formed so as to expand in a distal direction from the distal portion of the inner tube 20. The expandable portion 30 is stored in the outer tube 40 from the outer tube opening 41 while being elastically deformed and contracted as illustrated in FIG. 3 by operating the operating unit 50 to move the inner tube 20 in a proximal direction relatively with respect to the outer tube 40. In contrast, the expandable portion 30 is elastically expandable so as to project from the outer tube 40 in the distal direction and expand radially outward from the distal portion of the inner tube 20 as illustrated in FIGS. 1 and 2 by operating the operating unit 50 to move the inner tube 20 in the distal direction relatively with respect to the outer tube 40.

The detecting portion 70 includes a sensor 71 fixed to the expandable portion 30, and a signal cable 72 extending in the wiring lumen 22 from the operating unit 50 to the sensor 71. The configuration of the sensor 71 is not limited as long as a sensor can detect a contrast agent, and for example, an ultrasonic sensor, an infrared ray sensor, an optical sensor, a temperature sensor, and a viscosity sensor are applicable. The contrast agent used herein may be identified by X-rays, is used for intravascular administer, and is a compound containing ionic or non-ionic iodine atom having a molecular mass of not more than approximately 8000. More specifically, examples of the contrast agent include monomers such as iopromide, iopamidol, iomeprol, amidotrizoic acid, iohexol, iothalamic acid, iodamide, metrizoic acid, metorizoic amide, and ioxilan, and dimers such as ioxaglic acid, adipiodone, iotroxic acid, iodoxamic acid, and iotrolan. However, the contrast agent is not limited thereto.

The discharging portion 60 includes a discharging tube 61 disposed at the expandable portion 30 and configured to discharge a fluid, and a supply tube 62 configured to supply the fluid to the discharging tube 61. The discharging tube 61 is fixed to the expandable portion 30 so as to be wound around an outer peripheral surface of the expandable portion 30 by substantially 360 degrees. The discharging tube 61 communicates at one end thereof with the supply tube 62, and is closed at the other end. The discharging tube 61 includes a plurality of discharging holes 63 formed so as to be arranged along a winding direction (circumferential direction of the inner tube 20). The discharging holes 63 penetrate from an inner surface to an outer surface of the discharging tube 61, and all open toward the proximal direction. Since the plurality of discharging holes 63 are arranged in one direction (winding direction), if a fluid is discharged from the discharging holes 63, fluid parts discharged from the respective discharging holes 63 in the proximal direction are connected and form a film-shaped wall surrounding the outer peripheral surface of the inner tube 20. The discharging tube 61 is flexibly deformable, and is deformed by following the expandable portion 30 as the expandable portion 30 is contracted, and can be stored in the interior of the outer tube 40.

The supply tube 62 is a tubular member extending in the axial direction along outer surfaces of the inner tube 20 and the expandable portion 30, and includes a supply lumen 64 in the interior thereof. The supply lumen 64 of the supply tube 62 communicates at a distal portion thereof with a lumen of the discharging tube 61.

The material of the inner tube 20, the outer tube 40, and the supply tube 62 can be one of hard materials having flexibility and, for example, polyolefin such as polyethylene and polypropylene, polyester such as polyamide and polyethylene terephthalate, fluorinated polymer such as ETFE, PEEK (polyether ether ketone), and polyimide may also be used. A metallic blade or a coil may also be added to the above-described material in order to increase rigidity.

The material of the discharging tube 61 can be a flexible material and, for example, natural rubber, silicone rubber, nitrile rubber, and fluorine containing rubber may be used.

The material of the wire members 31 can be one of elastically deformable materials and, for example, shape-memory alloy which is provided with a shape-memory effect and superelasticity by heat treatment, metals such as stainless steel, tantalum, titanium, platinum, gold, and tungsten, polyolefin such as polyethylene and polypropylene, polyester such as polyamide and polyethylene terephthalate, fluorinated polymer such as ETFE, PEEK (polyether ether ketone), and polyimide may also be used. Among others, the shape-memory alloy can be used. As the shape-memory alloy, Ni—Ti based, Cu—Al—Ni based, Cu—Zn—Al based alloys can be used.

The length of the medical device 10 (the length of the expandable portion 30 from the distal most portion of the expandable portion 30 to the operating unit 50) is not limited, but can be included in a range, for example, from 500 mm to 1500 mm. The outer diameter of the outer tube 40 is not specifically limited, but can be included in a range from 3.0 mm to 5.0 mm. The outer diameter of the inner tube 20 is not specifically limited, but can be included in a range, for example, from 2.0 mm to 4.0 mm. The maximum outer diameter of the expandable portion 30 in a state in which the expandable portion 30 is expanded is not specifically limited as long as it is larger than the outer diameter of the descending aorta of a patient on an upstream of the entry portions of the renal arteries, but is can be included in a range, for example, from 20 mm to 40 mm.

The X-ray imaging marker 23 is mounted by winding a wire formed of the X-ray imaging material around an outer surface, or by forming a pipe with the X-ray imaging material and crimping or adhering the same on the outer surface. Examples of the X-ray imaging material can include, for example, gold, platinum, platinum-iridium alloy, silver, stainless, molybdenum, tungsten, tantalum, palladium, and alloy of these materials. Note that the X-ray imaging marker may be provided on the expandable portion 30, the outer tube 40 or on the discharging portion 60 instead of the inner tube 20.

The operating unit 50 is provided with the first operating unit 51 interlocked with a proximal end portion of the outer tube 40 and the second operating unit 52 interlocked with a proximal end portion of the inner tube 20. The inner tube 20 penetrates through the first operating unit 51 so as to be movable in the axial direction.

The second operating unit 52 includes an insertion port 53 communicating with the insertion lumen 21 of the inner tube 20 and a supply port 56 communicating with the supply lumen 64 of the supply tube 62. A connection cable 57 electrically connected to the signal cable 72 is drawn out from the second operating unit 52. The connection cable 57 can be connected to a detecting apparatus 90 configured to detect the concentration of the contrast agent upon reception of a signal from the sensor 71. The detecting apparatus 90 is connected to a control unit 92, and information on the concentration of the contrast agent detected by the detecting apparatus 90 is transmitted to the control unit 92.

The insertion port 53 allows insertion of the guide wire W. The supply port 56 can be connected to a fluid supply apparatus 91 configured to supply the fluid to the discharging tube 61 via the supply tube 62. The fluid supply apparatus 91 is, for example, a power injector. The fluid supply apparatus 91 is controlled by a control unit 92 and thus is capable of automatically controlling the supply of the fluid to the discharging portion 60. The control unit 92 is composed of, for example, a computer, which may have a processor and memory. The fluid to be supplied by the fluid supply apparatus 91 is not specifically limited as long as it is a fluid which can be discharged into blood vessels, and for example, physiological salt solution and carbon dioxide are applicable.

The material of the first operating unit 51 and the second operating unit 52 is not specifically limited, but for example, hard resins such as polycarbonate, polyethylene, polypropylene can be used.

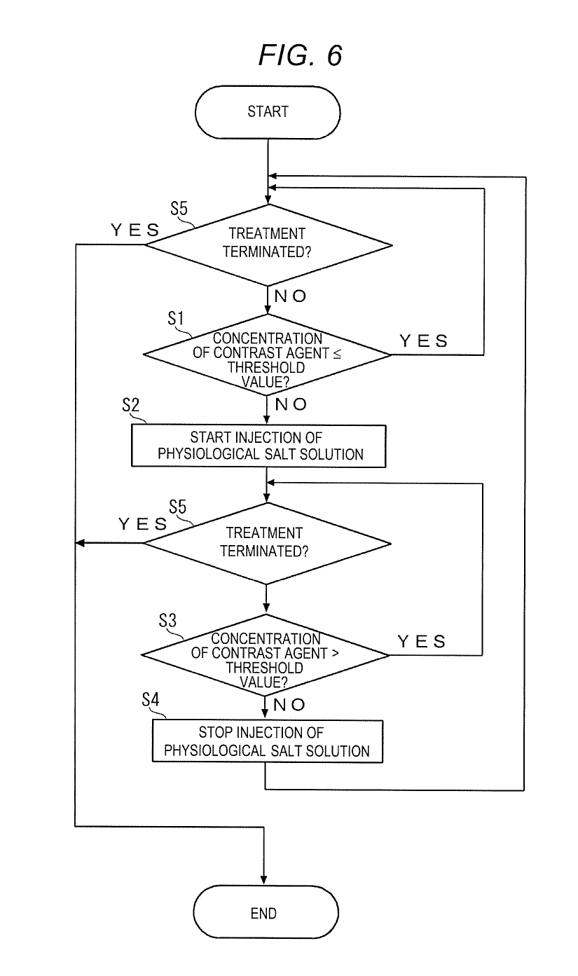

Next, a method of usage of the medical device 10 will be described with reference to a flowchart of the control unit illustrated in FIG. 6. Here, a case of suppressing an inflow of a contrast agent injected into coronary artery at the time of the PCI from descending aorta A to renal arteries R will be described. The fluid discharged from the discharging portion 60 is physiological salt solution.

Firstly, the medical device 10 to be used is primed, and the interior is substituted by physiological salt solution. In this initial state, the expandable portion 30 and the discharging tube 61 are stored and contracted in the outer tube 40 as illustrated in FIG. 3. The fluid supply apparatus 91 is then connected to the supply port 56 and the connection cable 57 is connected to the detecting apparatus 90 (see FIG. 1).

Next, an introducer sheath (not illustrated) is inserted into femoral artery. Next, the guide wire W is inserted into artery via the introducer sheath. Note that the position of installation of the introducer sheath is not limited as long as the medical device 10 can access the descending aorta A.

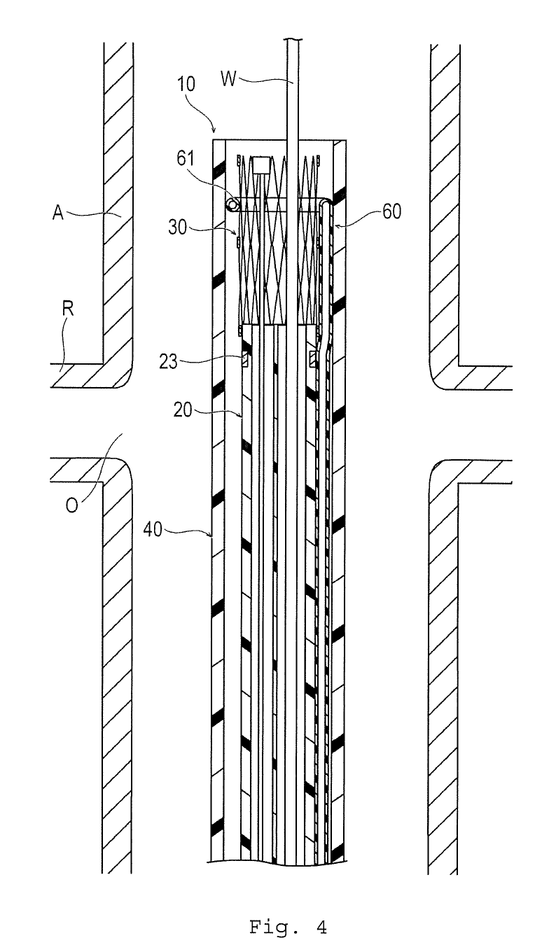

Next, the guide wire W is advanced to reach a distal side of entry portions O of the renal arteries R in the descending aorta A, that is, to an upstream side closer to the heart. Subsequently, the guide wire W is inserted into the insertion lumen 21 of the prepared medical device 10, and the medical device 10 is inserted into the artery along the guide wire W. Next, the medical device 10 is pushed and advanced along the guide wire W, so that the distal portion of the medical device 10 reaches the distal side of the entry portions O of the renal arteries R in the descending aorta A as illustrated in FIG. 4 (inserting step).

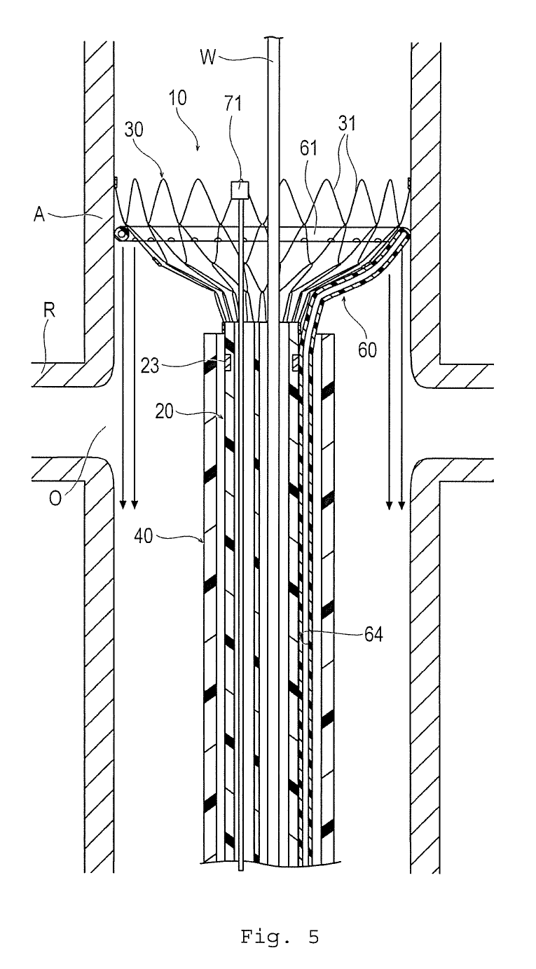

Next, if the first operating unit 51 is moved in the proximal direction with respect to the second operating unit 52 or if the second operating unit 52 is moved in the distal direction with respect to the first operating unit 51 while monitoring the position of the X-ray imaging marker 23 under radiography, the expandable portion 30 is moved in the distal direction from the outer tube 40 in a state in which the discharging portion 60 is positioned on the upstream of the entry portions O of the renal arteries R and is expanded to come into contact with an inner wall surface of the descending aorta A as illustrated in FIG. 5 (providing step). When the expandable portion 30 is expanded, blood flowing in the descending aorta A flows through the void portions of the wire member 31. When the expandable portion 30 is expanded, the discharging tube 61 fixed to the outer peripheral surface of the expandable portion 30 is also expanded, and is positioned at the proximity of the inner wall surface of the descending aorta A.

Next, the concentration of the contrast agent is detected by the detecting apparatus 90 on the basis of a signal from the sensor 71. The signal from the detecting apparatus 90 is input to the control unit 92, and whether the concentration of the contrast agent is not higher than a preset threshold value is determined (Step 51). In the case where the concentration of the contrast agent is not higher than the preset threshold value, supply of physiological salt solution from the fluid supply apparatus is not started.

Next, the contrast agent is injected into coronary artery by another catheter inserted from radial artery into artery for the PCI. Note that the another catheter does not pass the vicinity of the entry portions O of the renal arteries R in the descending aorta A, and thus does not overlap with the medical device 10 in position. The contrast agent injected into the coronary artery runs through coronary vein and reaches coronary sinus, further runs in the heart from right atrium, and reaches the descending aorta A. If the concentration of the contrast agent detected by the detecting apparatus 90 on the basis of the signal from the sensor 71 exceeds the preset threshold value, the control unit 92 that has received a signal from the detecting apparatus 90 automatically controls the fluid supply apparatus 91, and supplies physiological salt solution to the discharging tube 61 via the supply port 56 and the supply lumen 64 (Step S2). The physiological salt solution supplied to the discharging tube 61 is discharged from the plurality of discharging holes 63 arranged in one direction (the winding direction, the circumferential direction of the shaft portion), and fluid portions discharged from the respective discharging holes 63 in the proximal direction are joined and flow in a form of a film-shaped wall along the inner wall surface of the descending aorta A and covers the entry portions O of the renal arteries R. At this time, with the discharging holes 63 arranged in the circumferential direction of the inner tube 20 (shaft portion), the film-shaped flow of the physiological salt solution can be easily formed so as to extend along the blood vessel wall. Accordingly, the inflow of the contrast agent flowing in the descending aorta A into the renal arteries R is reduced, and the contrast agent having a high concentration is diluted by the physiological salt solution. Therefore, the amount and the concentration of the contrast agent in blood running through the renal arteries R and reaching kidney reduce and thus an effect of the contrast agent on kidney reduces. Since the expandable portion 30 is in contact with the intravascular wall surface and the position of the inner tube 20 is fixed, the discharging direction of physiological salt solution is stabilized, and thus the inflow of the contrast agent into the renal arteries R can be reduced further effectively.

Since the discharging holes 63 are provided over an entire circumference in a line, the film shaped flow is also formed over the entire circumference. Therefore, adjustment of the position of the medical device 10 in a direction of rotation is not necessary, and thus operation is easy. Note that even though the discharging holes 63 are arranged in line over the entire circumference, blood and the contrast agent can flow inside the film-shaped flow formed over the entire circumference, and thus the flows of the blood and the contrast agent directed toward the arteries of the lower limbs are not hindered.

Whether the concentration of the contrast agent detected by the detecting apparatus 90 on the basis of a signal from the sensor 71 is not higher than the threshold value is always, or at least periodically, determined (Step S3). If the concentration of the contrast agent is lowered to a level equal to or lower than the threshold value, the control unit 92 that has received the signal from the detecting apparatus 90 automatically controls the fluid supply apparatus 91, and stops the supply of the physiological salt solution (Step S4). Accordingly, discharge of the physiological salt solution from the discharging holes 63 stops and the inflow of blood in the descending aorta A which contains little contrast agent into the renal arteries R is enabled without being hindered by the physiological salt solution.

The medical device 10 then operates in accordance with administration of the contrast agent into the coronary artery, and discharges physiological salt solution from the discharging holes 63 to suppress the inflow of the contrast agent into the renal arteries R only when the concentration of the contrast agent in the blood exceeds the threshold value.

After the completion of the procedure of the PCI, the fluid supply apparatus 91 and the detecting apparatus 90 are stopped (Step S5). Subsequently, if the first operating unit 51 is moved in the distal direction with respect to the second operating unit 52 or if the second operating unit 52 is moved in the proximal direction with respect to the first operating unit 51, the expandable portion 30 and the discharging tube 61 are stored and are contracted in the outer tube 40 as illustrated in FIG. 4 (contracting step).

Subsequently, the medical device 10 and the guide wire W are pulled out from the introducer sheath, and the introducer sheath is removed from a vein V to complete the treatment.

As described above, the medical device 10 according to the first embodiment is the medical device 10 configured to suppress the inflow of a contrast agent into kidney, includes the elongated inner tube 20 (shaft portion) and the discharging portion 60 configured to discharge a fluid in the proximal direction from both sides of the distal portion of the inner tube 20 with the axial center interposed therebetween, and the discharging portion 60 includes the plurality of discharging holes 63 arranged in one direction. The medical device 10 configured as described above discharges a fluid from the plurality of discharging holes 63 arranged in one direction, and thus is capable of discharging the fluid as a film-shaped wall. With the configuration in which the film-shaped fluid is discharged from both sides of the inner tube 20 with the axial center interposed therebetween, the fluid can be flowed so as to block off both of the entry portions O of the two renal arteries R branched from the descending aorta A in which the inner tube 20 is inserted, so that inflow of the contrast agent to the renal arteries R is effectively reduced, and the contrast agent can be diluted. Consequently, an effect of the contrast agent on kidney reduces. Since the medical device 10 does not contact with an endothelium of the renal artery, stenosis of the renal artery reduces. Note that the flow in the proximal direction in this embodiment does not strictly have to be a flow parallel to the axial center of the inner tube 20 (shaft portion),and means a flow inclined toward the proximal side with respect to a direction orthogonal to the axial center of the inner tube 20 (shaft portion) (a direction toward the blood vessel wall).

Since the inner tube 20 (shaft portion) includes the expandable portion 30 configured to expand radially outward, the position of the inner tube 20 can be fixed by bringing the expandable portion 30 into contact with an intravascular wall surface, so that the inflow of the contrast agent into the renal arteries R is reduced further effectively with a stable fluid discharging direction.

Since the discharging portion 60 is disposed at the expandable portion 30, the fluid can be discharged so as to flow from a position close to the blood vessel wall along the blood vessel wall, so that the inflow of the contrast agent into the renal arteries R is reduced further effectively by the film-shaped fluid.

Since the medical device 10 further includes the detecting portion 70 configured to detect the contrast agent, the fluid can be discharged at the same timing as the contrast agent is detected, so that the inflow of the contrast agent into the renal arteries R is reduced further effectively.

Since the inner tube 20 includes the X-ray imaging marker 23 at the distal portion, the inner tube 20 can be disposed at an adequate position under radiography, and discharge of the fluid to a desirable position is enabled, so that the inflow of the contrast agent into the renal arteries R is reduced further effectively.

Further, a treatment (remedy) method for suppressing an inflow of a contrast agent into kidney is also provided. The aforesaid treatment method includes: (i) an inserting step for inserting a medical device having a discharging portion configured to be capable of discharging a fluid at a distal portion of an elongated shaft portion into descending aorta; (ii) a providing step for providing the discharging portion on an upstream of an entry portion of renal artery; and (iii) a discharging step for discharging the fluid from the discharging portion in a film shape so as to cover the entry portion upon arriving of the contrast agent flowing from the upstream at the entry portion of the renal artery. According to the aforesaid treatment method, since the fluid is discharged in a film shape so as to cover the entry portion of the renal artery, the inflow of the contrast agent reaching the entry portion of the renal artery into renal artery is further effectively reduced by the film-shaped fluid while diluting the contrast agent with the fluid. By reducing the inflow of the contrast agent having a high concentration into the renal arteries, an effect of the contrast agent on kidney is reduced.

According to the aforesaid treatment method, in the discharging step, a fluid is discharged from the discharging portion in the direction orthogonal to the shaft center or in the proximal direction from both sides of the distal portion of the shaft portion with the axial center being interposed between the both sides of the distal portion. Therefore, generation of the film-shaped flow so as to block off both of the entry portions of the two renal arteries branched from the descending aorta is enabled, and the inflow of the contrast agent into the renal arteries is reduced.

According to the aforesaid treatment method, in the providing step, the position of the shaft portion is fixed by expanding the expandable portion configured to be capable of expanding radially outward in both directions from the distal portion of the shaft portion and bringing the expandable portion into contact with the descending aorta. Therefore, by fixing the position of the shaft portion by the expandable portion, the inflow of the contrast agent into renal arteries is further effectively reduced by stabilizing the fluid discharging direction.

According to the aforesaid treatment method, in the providing step, fluid is discharged from the discharging portion provided at the expandable portion. Therefore, the fluid can be discharged so as to flow along the blood vessel wall from a position close to the blood vessel wall, so that the inflow of the contrast agent into the renal arteries is reduced further effectively by the film-shaped fluid.

According to the aforesaid treatment method, in the discharging step, the fluid is discharged from the discharging portion when the contrast agent is detected by the detecting portion configured to be capable of detecting the contrast agent and provided on the medical device. Therefore, the fluid can be discharged at the same timing as the contrast agent is detected, so that the inflow of the contrast agent into the renal arteries is reduced further effectively.

According to the aforesaid treatment method, in the providing step, the discharging portion is provided while confirming the position of the X-ray imaging marker provided at the distal portion of the medical device under radiography. Therefore, the discharging portion can be provided at an adequate position and thus the fluid can be discharged to a desired position, so that the inflow of the contrast agent into the renal arteries is reduced further effectively.

Note that in the example described above, in the case where the concentration of the contrast agent detected by the sensor 71 exceeds a threshold value, the control unit 92 controls the supply apparatus to discharge physiological salt solution. However, the configuration is not limited thereto. For example, after the injection of the contrast agent into the coronary artery, the control unit 92 may be controlled to discharge physiological salt solution by operating the fluid supply apparatus 91 at a predetermined time for a predetermined period by setting a timer. It is also possible to confirm the contrast agent under radiography and to discharge physiological salt solution by a manual operation. In this configuration, the sensor 71 for detecting the contrast agent does not have to be provided. The structure of the expandable portion is not limited to the structure having the wire portions as long as it can be expanded and can be brought into contact with the descending aorta, and may be, for example, a balloon. Note that when the balloon is employed as the dilation portion, the shape of the balloon should not block off the descending aorta A completely when the balloon is dilated.

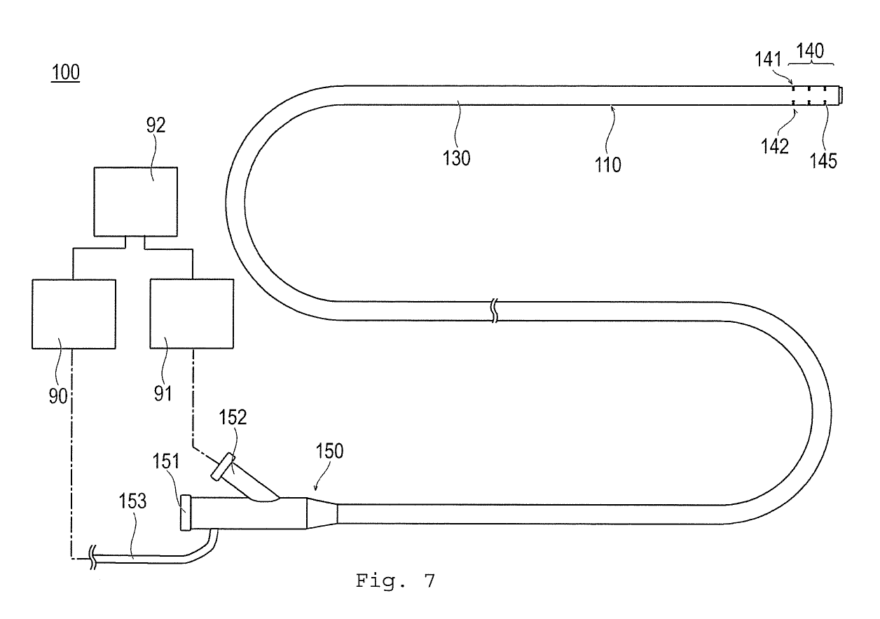

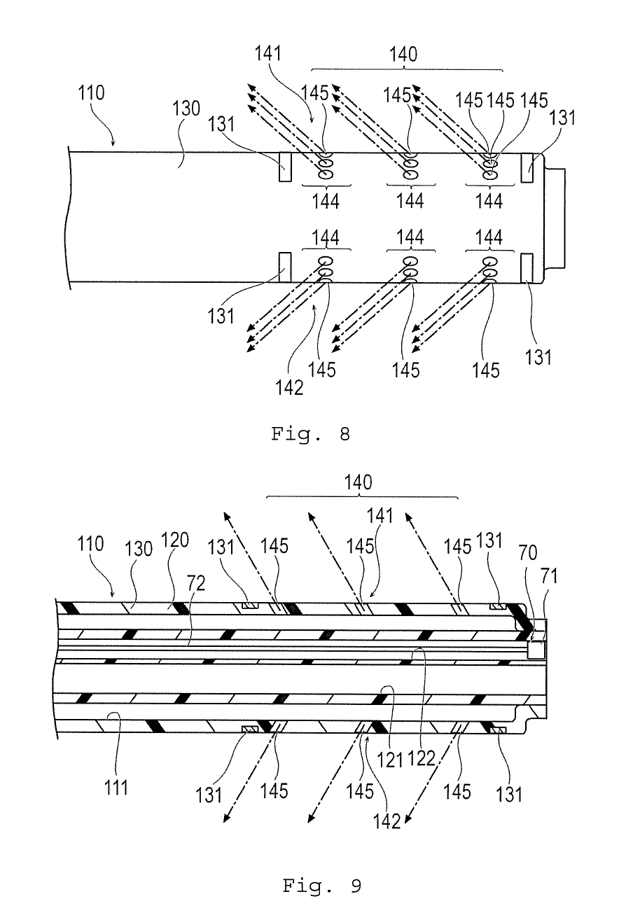

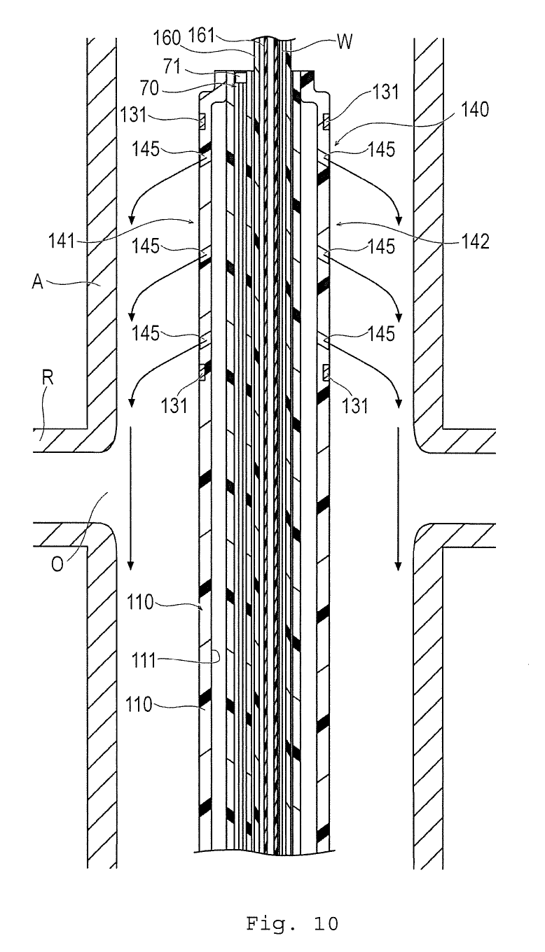

Another configuration for the medical device 100 may not provide the expandable portion, as illustrated in FIGS. 7 to 9. Note that portions having the same functions as those described above are denoted by the same reference numerals and that description is omitted.

A medical device 100 includes a shaft portion 110, a discharging portion 140 configured to discharge a fluid, the detecting portion 70 configured to detect a contrast agent, and an operating unit 150, as illustrated in FIGS. 7 to 9. The shaft portion 110 includes an inner shaft 120 which is an elongated tubular member, and an outer shaft 130 which is an elongated tubular member and configured to allow storage of the inner shaft 120 in the interior thereof

The inner shaft 120 and the outer shaft 130 are interlocked with each other at distal portions thereof to form a duplicated tube structure, and a supply lumen 111 is formed between the inner shaft 120 and the outer shaft 130. Proximal portions of the inner shaft 120 and the outer shaft 130 are interlocked with the operating unit 150.

The inner shaft 120 includes an insertion lumen 121 formed to allow insertion of a guide wire W in the interior thereof, and a wiring lumen 122 in which the detecting portion 70 is disposed. The insertion lumen 121 and the wiring lumen 122 open at a distal end portion of the inner shaft 120.

The discharging portion 140 including a plurality of discharging holes 145 penetrating from an inner surface to an outer surface is provided at a distal portion of the outer shaft 130. The discharging portion 140 is formed separately into a first discharging portion 141 and a second discharging portion 142 positioned on both sides of the outer shaft 130 with an axial center interposed therebetween. Each of the first discharging portion 141 and the second discharging portion 142 includes three rows of discharging hole groups 144 in the axial direction each including the discharging holes 145 arranged in the circumferential direction. The discharging holes 145 are formed so as to be inclined toward the proximal direction with respect to a direction orthogonal to the axial center of the outer shaft 130. X-ray imaging markers 131 are provided at the distal portion of the outer shaft 130 at positions corresponding to a proximal side and a distal side of the discharging portion 140 so as to achieve easy identification of the position of the discharging portion 140 under radiography. In addition, the X-ray imaging markers 131 are arranged separately at two positions in the circumferential direction corresponding to positions of the first discharging portion 141 and the second discharging portion 142 with the axial center of the shaft portion 110 being interposed therebetween.

Since the plurality of discharge holes 145 which constitute each of the discharging hole groups 144 are formed in a line in one direction (circumferential direction), when the discharging holes 145 discharge a fluid, the fluid parts discharged respectively from the discharging holes 145 in the proximal direction are joined to form a film-shaped wall.

The material of the inner shaft 120 and the outer shaft 130 can be one of hard materials having flexibility and, for example, polyolefin such as polyethylene and polypropylene, polyester such as polyamide and polyethylene terephthalate, fluorinated polymer such as ETFE, PEEK (polyether ether ketone), and polyimide may also be used. A metallic blade or a coil may be added to the above-described material to increase rigidity.

The operating unit 150 includes an insertion port 151 communicating with the insertion lumen 121 and a supply port 152 communicating with the supply lumen 111. A connection cable 153 electrically connected to a signal cable 72 located in the wiring lumen 122 is drawn out from the operating unit 150.

The insertion port 151 allows insertion of the guide wire W, a guiding catheter 160, and a catheter 161 described later. The supply port 152 can be connected to the fluid supply apparatus 91 for supplying a fluid to the discharging holes 145 via the supply lumen 111.

Next, a method of using the reconfigured medical device 100 according will be described. Here, a case of suppressing an inflow of a contrast agent injected into coronary artery at the time of the PCI from descending aorta A to renal arteries R will be described as an example. The fluid flowing out from the discharging portion 140 is physiological salt solution.

Firstly, the medical device 100 to be used is primed, and the interior is substituted by physiological salt solution. In this initial state, the fluid supply apparatus 91 is connected to the supply port 152 and the connection cable 153 is connected to the detecting apparatus 90 (see FIG. 7).

Next, an introducer sheath (not illustrated) is inserted into femoral artery. Next, the guide wire W is inserted into artery via the introducer sheath. Note that the position where the introducer sheath is installed is not limited as long as the medical device 100 can access the descending aorta A.

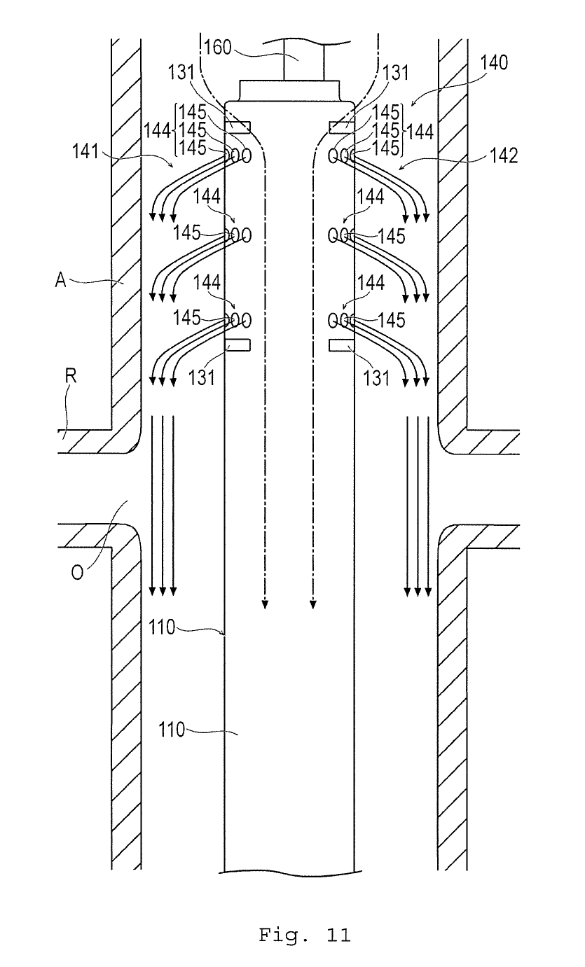

Next, the guide wire W is advanced to reach a distal side of entry portions O of the renal arteries R in the descending aorta A, that is, to an upstream side closer to the heart. Subsequently, the guide wire W is inserted into the insertion lumen 121 of the prepared medical device 100, and the medical device 100 is inserted into the artery along the guide wire W. Next, the medical device 100 is pushed and advanced along the guide wire W, so that the distal portion of the medical device 100 reaches the distal side of the entry portions O of the renal arteries R in the descending aorta A as illustrated in FIGS. 10 and 11 (inserting step). The first discharging portion 141 and the second discharging portion 142 are then provided on the upstream of the two entry portions O while confirming the position of the X-ray imaging markers 131 under radiography (providing step).

Next, the concentration of the contrast agent is detected by the detecting apparatus 90 on the basis of a signal from the sensor 71. The signal from the detecting apparatus 90 is input to the control unit 92, and whether the concentration of the contrast agent is not higher than a preset threshold value is determined. In the case where the concentration of the contrast agent is not higher than the preset threshold value, supply of physiological salt solution from the fluid supply apparatus is not started.

Next, the guiding catheter 160 for performing the procedure of the PCI and the catheter 161 (for example, a balloon catheter) for treatment that passes through the interior of the guiding catheter 160 are inserted from the insertion port 151 of the operating unit 150 along the guide wire W to reach coronary artery for the PCI. Subsequently, a contrast agent is injected into coronary artery via the guiding catheter 160. The contrast agent injected into the coronary artery runs through coronary vein and reaches coronary sinus, further runs in heart from right atrium, and reaches the descending aorta A. If the concentration of the contrast agent detected by the detecting apparatus 90 on the basis of the signal from the sensor 71 exceeds the threshold value, the control unit 92 that has received a signal from the detecting apparatus 90 automatically controls the fluid supply apparatus 91, and supplies physiological salt solution to the discharging portion 140 via the supply port 152 and the supply lumen 111. The physiological salt solution supplied to the discharging portion 140 is discharged from the discharging hole groups 144 provided respectively on the first discharging portion 141 and the second discharging portion 142. Since the discharging hole groups 144 each include the plurality of discharging holes 145 arranged in a line, the physiological salt solution parts discharged in the proximal direction are joined to form a film-shaped wall which directs toward the inner wall surface of the descending aorta A. The physiological salt solution in contact with the inner wall surface of the descending aorta A redirects the flowing direction, flows downstream as the film-shaped wall along the inner wall surface, and covers the entry portions O of the renal arteries R. Accordingly, the inflow of the contrast agent flowing in the descending aorta A into the renal arteries R is reduced, and the contrast agent is diluted by the physiological salt solution. Therefore, the amount and the concentration of the contrast agent in blood running through the renal arteries R and reaching kidney reduce, and thus an effect of the contrast agent on kidney reduces. Since the first discharging portion 141 and the second discharging portion 142 each include a plurality (three in this embodiment) of the discharging hole groups 144, an effect of suppressing the inflow of the contrast agent into the renal arteries R and an effect of diluting the contrast agent are increased. Since there is an area from which the physiological salt solution is not discharged between the first discharging portion 141 and the second discharging portion 142, flows of the contrast agent and blood in the descending aorta A can proceed downstream of the entry portions O of the renal arteries R while avoiding the wall of the physiological salt solution as illustrated by arrows of a dot-and-dash line in FIG. 11.

Whether the concentration of the contrast agent detected by the detecting apparatus 90 on the basis of a signal from the sensor 71 is not higher than the threshold value is always, or at least periodically, determined. If the concentration of the contrast agent is lowered to a level equal to or lower than the threshold value, the control unit 92 that has received the signal from the detecting apparatus 90 automatically controls the fluid supply apparatus 91, and stops the supply of the physiological salt solution. Accordingly, discharge of the physiological salt solution from the discharging holes 145 stops and blood in the descending aorta A containing little contrast agent flows into the renal arteries R without being hindered by the physiological salt solution.

The reconfigured medical device 100 then operates in accordance with administration of the contrast agent into the coronary artery, and discharges physiological salt solution from the discharging holes 145 only when the concentration of the contrast agent in the blood exceeds the threshold value to suppress the inflow of the contrast agent into the renal arteries R.

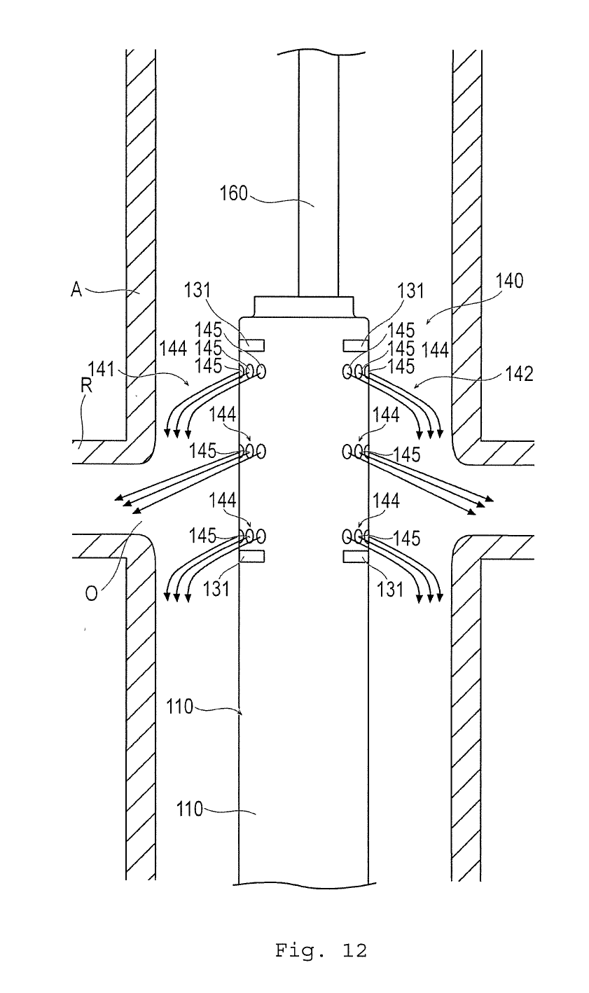

After the completion of the procedure of the PCI, the reconfigured medical device 100 is moved to the proximal side (downstream side) to a position where the physiological salt solution discharged from the discharging holes 145 flows directly into the renal arteries R while monitoring the X-ray imaging markers 131 and the physiological salt solution is discharged from the discharging holes 145 as illustrated in FIG. 12. Accordingly, the interior of the renal arteries R is cleaned and further reduction of an effect of the contrast agent on the renal arteries R is achieved.

Subsequently, the catheter 161, the guiding catheter 160, the medical device 100 and the guide wire W are pulled out from the introducer sheath, and the introducer sheath is removed from a blood vessel to complete the treatment.

As described thus far, the reconfigured medical device 100 is not provided with the expandable portion, and a fluid from the discharging portion 140 provided at the shaft portion 110 is discharged. In this configuration as well, the fluid can be flowed in a film shape so as to block off both of the entry portions O of the two renal arteries R branched from the descending aorta A into which the shaft portion 110 is inserted. Therefore, the inflow of the contrast agent into the renal arteries R is effectively reduced, and the contrast agent is diluted by the fluid, so that an effect of the contrast agent on kidney is reduced.

Note that the claims are not limited only to the embodiments described above, and various modifications may be made by those skilled in the art within a technical scope of the embodiments. For example, the position where the contrast agent is administered is not limited to coronary artery. For example, left ventriculography of heart and aortic angiography in Transcatheter Aortic Valve Implantation (TAVI) are also included.

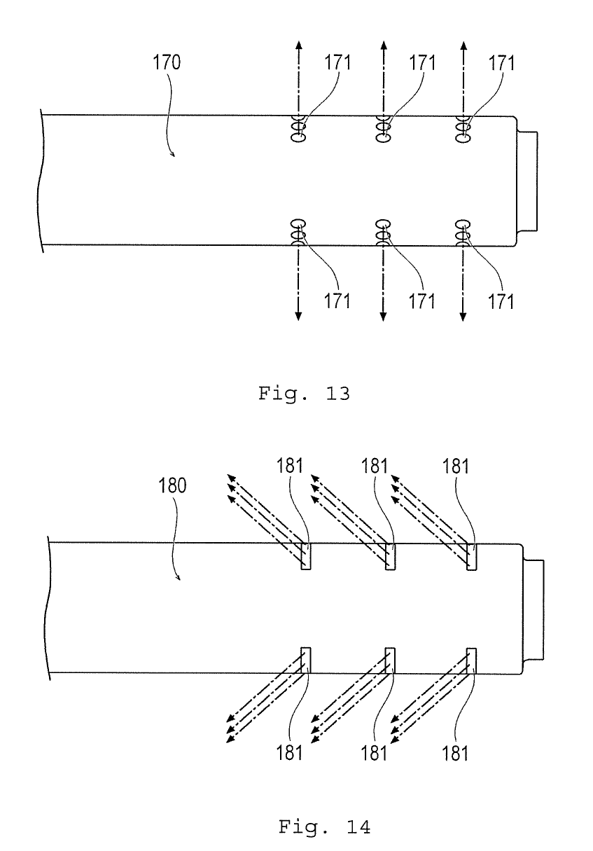

An additional or alternative configuration of the medical device 100 may be as illustrated in FIG. 13, where discharging holes 171 may be formed on a shaft portion 170 and may be formed in a direction orthogonal to an axial center of the shaft portion 170. In this configuration, fluid discharged from the discharging holes 171 hits against the blood vessel wall and becomes a film-shaped flow flowing along the blood vessel wall to cover the entry portions O of the renal arteries R.

Another additional or alternative configuration of the medical device 100 may be illustrated in FIG. 14, where discharging holes 181 can be formed on a shaft portion 180 and may be formed so as to be elongated in one direction (circumferential direction) in a slit shape. In this configuration, the fluid discharged from the discharging holes becomes a film shape and thus the fluid can be flowed so as to block off the entry portions O of the renal arteries R, so that the inflow of the contrast agent into the renal arteries R is effectively reduced.

In addition, the guiding catheter 160 and the catheter 161 may be inserted into the insertion lumen 21 of the medical device 10. Also, the guiding catheter 160 and the catheter 161 may not be inserted into the insertion lumen 121 of the medical device 100.

### DESCRIPTION OF REFERENCE SIGNS

10, 100 medical device;

20 inner tube (shaft portion);

30 expandable portion;

110, 170, 180 shaft portion;

131 X-ray imaging marker;

60, 140 discharging portion;

63, 145, 171, 181 discharging holes;

70 detecting portion;

A descending aorta;

O entry portion;

R renal artery.

What is claimed is:

1. A medical device for suppressing an inflow of a contrast agent into kidney, comprising:
an elongated shaft portion;
a discharging portion configured to be capable of discharging a fluid in a direction orthogonal to an axial center or in a proximal direction from both sides of a distal portion of the shaft portion with the axial center being interposed between the both sides of the distal portion, wherein the discharging portion includes one of:
a plurality of discharging holes arranged in one direction; or
a plurality of discharging holes elongated in one direction in a slit shape.

2. The medical device according to claim 1, wherein the shaft portion includes an expandable portion configured to be capable of expanding radially outward.

3. The medical device according to claim 2, wherein the discharging portion is disposed at the expandable portion.

4. The medical device according to claim 3, further comprising a detecting portion configured to detect the contrast agent.

5. The medical device according to claim 4, wherein the shaft portion includes an X-ray imaging marker at the distal portion thereof

6. The medical device according to claim 3, wherein the shaft portion includes an X-ray imaging marker at the distal portion thereof.

7. The medical device according to claim 2, further comprising a detecting portion configured to detect the contrast agent.

8. The medical device according to claim 2, wherein the shaft portion includes an X-ray imaging marker at the distal portion thereof.

9. The medical device according to claim 1, further comprising a detecting portion configured to detect the contrast agent.

10. The medical device according to claim 1, wherein the shaft portion includes an X-ray imaging marker at the distal portion thereof

11. A treatment method for suppressing an inflow of a contrast agent into kidney, comprising:
inserting a medical device having a discharging portion configured to be capable of discharging a fluid at a distal portion of an elongated shaft portion into descending aorta;
providing the discharging portion on an upstream of an entry portion of a renal artery; and
discharging the fluid from the discharging portion in a film shape so as to cover the entry portion upon arriving of the contrast agent flowing from the upstream at the entry portion of the renal artery.

12. The treatment method according to claim 11, wherein the discharging step includes discharging the fluid from a discharging portion in a direction orthogonal to a shaft center or in a proximal direction from both sides of a distal portion of the shaft portion with the axial center being interposed between the both sides of the distal portion.

13. The treatment method according to claim 12, wherein the providing step includes:
fixing a position of the shaft portion by expanding an expandable portion configured to be capable of expanding radially outward in both directions from a distal portion of the shaft portion; and
bringing the expandable portion into contact with the descending aorta.

14. The treatment method according to claim 13, wherein the providing step includes discharging the fluid from the discharging portion provided at the expandable portion.

15. The treatment method according to claim 14, wherein the discharging step includes discharging the fluid from the discharging portion when the contrast agent is detected by a detecting portion configured to be capable of detecting the contrast agent and provided on the medical device.

16. The treatment method according to claim 15, wherein the providing step includes providing the discharging portion while confirming a position of an X-ray imaging marker provided at the distal portion of the medical device under radiography.

17. The treatment method according to claim 16, wherein the fluid is discharged from a plurality of discharging holes arranged in one direction.

18. The treatment method according to claim 17, wherein the fluid is discharged from a plurality of discharging holes elongated in one direction in a slit shape.

19. The treatment method according to claim 18, wherein the plurality of holes are arranged on only a portion of a radial portion of the elongated shaft portion.

20. The treatment method according to claim 19, wherein the plurality of holes are arranged in at least two radial portions.
